(19) United States
(12) Patent Application Publication
(10) Pub. No.: US 2009/0064999 A1
(43) Pub. Date: Mar. 12, 2009

(54) Tracheostomy tube with inner cannula and obturator for regular and extra-long stomas

(75) Inventors: Lewis H. Marten, Westwood, MA (US); Dennis F. Creedon, Sandwich, MA (US)

Correspondence Address: FROMMER LAWRENCE & HAUG, 745 FIFTH AVENUE- 10TH FL., NEW YORK, NY, 10151, US

(73) Assignee: E. Benson Hood Laboratories

(21) Appl. No.: 11/900,470

(22) Filed: Sep. 12, 2007

(51) Int. Cl. A61M 16/04

(52) U.S. Cl. 128/200.26

(57) ABSTRACT

This invention relates to a dual-cannula tracheostomy device comprising an outer cannula and an inner cannula for insertion into the trachea of patients with normal-sized and extra-long stomas. The inner cannula comprises an interlocking means which interacts with a connecting means on the outer cannula in order to stabilize the inner cannula when it is inserted in the outer cannula. The inner cannula may also comprise a gripping means, such as ridges, which improve handling of the inner cannula during its insertion and removal from the outer cannula. The invention further comprises an obturator to help guide the outer cannula while it is being inserted into the trachea. The obturator similarly comprises an interlocking means which interacts with the connecting means on the outer cannula to stabilize the obturator while it is inserted in the outer cannula. Finally, the invention comprises a method of inserting the tracheostomy device into the trachea of a patient.

### INCORPORATION BY REFERENCE

All patents, patent applications, documents and/or references referred to herein are incorporated by reference, and may be employed in the practice of the invention.

### FIELD OF THE INVENTION

This invention relates to a tracheostomy device comprising an outer cannula, an inner cannula, and an obturator for insertion into the trachea of patients with normal-sized and extra-long stomas. Specifically, this invention is directed to an inner cannula and obturator having a means for easing handling during insertion and removal from the outer cannula, and an improved means of interlocking the inner and outer cannula and interlocking the obturator and outer cannula together.

### BACKGROUND OF THE INVENTION

Tracheostomy is a common procedure used to open a direct airway through an incision in the trachea. The procedure is often performed when the need arises for prolonged airway support in patients with respiratory failure, as it allows for a more secure and manageable airway, safer enteral feeding, and better overall management of the patient. A tracheostomy is also performed in other situations such as for sleep apnea related to obstruction from obesity, congenital abnormality of the larynx or trachea, severe neck or mouth injuries or cancer, inhalation injuries, presence of a large foreign obstruction in the airway, vocal cord paralysis that affects swallowing and increases risk of aspiration, inability to clear secretions, and tracheal stenosis or malacia.

Tracheostomy involves the placement of a tube into the trachea, which is a cartilaginous tubular structure in the throat comprised of 18 to 22 C-shaped rings of rigid cartilage oriented anteriorly and laterally, and a membranous posterior portion. There are two types of tracheostomy techniques: surgical tracheostomy and percutaneous tracheostomy. Surgical tracheostomy typically involves the placement of the tube in the region of the second to fourth tracheal rings. During this procedure, an anterior part of one of the tracheal rings is removed to either produce a stoma, or create a flap with the severed part of the rings. In contrast, percutaneous tracheostomy involves the placement of a tracheostomy tube between the first and second, or between the second and third tracheal rings. This technique involves placing a guide wire between the tracheal rings, and sequentially forcing a series of stiff plastic dilators into the tracheal wall over the guide wire until a stoma is created that can sufficiently accommodate the tracheostomy tube.

Tracheostomy tubes are used to administer positive-pressure ventilation, to protect against aspiration, to provide an airway in patients prone to upper airway obstruction, and to provide access to the lower respiratory tract for airway clearance. Tubes can vary in rigidity, internal and external diameter, and cuff design. Rigidity is dependent on the material of the tube, although more rigid tubes may have a greater tendency to cause mucosal trauma and an increased risk of perforating the tracheal wall. The cuff is a balloon-like device around the distal end of the tracheostomy tube, which can be inflated to protect the airway; this is useful, for example, in patients who are at risk of aspirating.

Dual-cannula tracheostomy devices are designed to be used with an inner cannula. The inner cannula is inserted into the tube or outer cannula, and is used to maintain the airway provided by the outer cannula as clean. The inner cannula may be disposable or may be reusable such that it is cleaned or replace periodically with a new inner cannula. It is believed that cleaning or replacement may reduce biofilm formation and the incidence of ventilator-associated pneumonia. The inner cannula can be a low-profile inner cannula which is used for spontaneous breathing, or an inner cannula with a connector to attach a ventilator.

For insertion of the dual-cannula tracheostomy device into the airway, an obturator is first placed inside the outer cannula to provide rigid support. The outer cannula is then inserted through the stoma and into the airway. Once the outer cannula is in place, the obturator is removed. An inner cannula is then inserted into the outer cannula, and once the lumen becomes compromised by a build-up of mucus and other deposits, the inner cannula can be replaced with a new inner cannula.

Importantly, there are problems associated with tracheostomy devices due to their design. For instance, the obturator or inner cannula may move or change position while inserted into the outer cannula. In addition, there is often no convenient means of handling the obturator or inner cannula during insertion and removal from the outer cannula.

There are few patented devices associated with tracheostomy tubes that are directed to improving the positioning of inner cannula/obturator or providing easier insertion or removal of the cannula/obturator. U.S. Pat. No. 3,659,612 relates to a tracheostomy tube comprising an outer and inner cannula, wherein the outer cannula and inner cannula are sealed together by attachment of a removal fitting which partially fits over the outer and inner cannula. Unfortunately, the attachment and detachment procedure of the inner cannula to the outer cannula are not simple to perform and the inner cannula is not easy to handle. Other patents such as U.S. Pat. Nos. 3,639,624, 4,009,720, 4,135,505, 5,067,515, and 5,067,496 provide a means of clamping the inner cannula to the outer cannula, although this type of engagement can be accidentally disconnected by the patient. U.S. Pat. No. 5,460,176 relates to a device comprising an outer cannula, an inner cannula, a connector head that circumscribes the end of the outer cannula, a connector body that links to the inner cannula and is locked to the connector head, and a securing component that is fitted over the interlocked connector head and body. While this secures the inner and outer cannulas, it does not provide an easy means of removing the inner cannula from the outer cannula and requires the manipulation and handling of multiple components. Furthermore, U.S. Pat. No. 6,481,436 relates to an obturator and tracheostomy tube, wherein the end of the obturator that is towards the outside of the patient's body has a cruciform section with a catch that snaps onto the lip-like edge of the flange on the tracheostomy tube. The obturator is removed by twisting the flange about 20° so that the catch comes out of engagement with the lip-like edge. This type of design, however, can lead to accidental disengagement of the obturator from the tube if the flange or obturator is incidentally twisted during the insertion of the tube into the trachea. Clearly, there is not an effective design for a tracheostomy device wherein the obturator and inner cannula can be sufficiently locked to the outer cannula and manageably inserted and removed from the outer cannula.

The problem of having an ineffectively designed tracheostomy devices is especially important for tracheostomy devices inserted into obese patients. Obese patients typically encounter difficulties when using an average shape/size tracheostomy device, as the tracheal tube/outer cannula does not match the anatomy of the stoma in an obese patient. The tracheal stoma in such patients is longer due to the increased distance between the exterior of the neck and the anterior tracheal wall. Difficulties associated with inefficiently designed tracheostomy device include tube impingement on the posterior tracheal wall, blood clots, mucous plugs, and partial displacement. Patients with a long stoma require a tracheostomy device with a shape that reflects the anatomy of the stoma and the extra distance between the proximal stoma opening and the anterior tracheal wall. The radius of the resultant outer cannula is not constant, posing a geometry that makes the introduction of a typical obturator and inner cannula difficult. Both components would need to be flexible in order to travel through the lumen of the extra-long outer cannula.

Citation or identification of any document in this application is not an admission that such document is available as prior art to the present invention.

### SUMMARY OF THE INVENTION

The present invention is directed to a dual-cannula tracheostomy device that provides an improved means of stabilizing the inner cannula when it is inserted into the outer cannula tracheostomy tube, and an improved means of handling the inner cannula during its insertion and removal from the outer cannula.

Another aspect of the invention is an improved means of stabilizing the obturator when it is inserted into the outer cannula tracheostomy tube, and an improved means of handling the obturator during its insertion and removal from the outer cannula.

Yet another aspect of the invention is directed to a dual-cannula tracheostomy device for insertion into patients having extra-long tracheal stomas. This device has a means of locking the inner cannula and/or obturator when it is inserted into the outer cannula, and a means of handling the inner cannula and/or obturator during its insertion and removal from the outer cannula.

Another aspect of the invention is a method of inserting a tracheostomy device into the trachea of a patient having a normal or extra-long stoma.

The present invention may comprise an outer cannula and a removable inner cannula. The outer cannula may comprise a distal end that is inserted into the trachea, a proximal end that is outside of the trachea, and a middle lumen therebetween which may have a horizontal section adjacent proximally to a section having a general curvature. The inner cannula may comprise a distal end that is inserted into the outer cannula, a proximal end that is outside of the trachea, and a middle region therebetween which also has a horizontal section proximally adjacent to a section having a general curvature. The inner cannula features a gripping means on its proximal end, which enables ease in handling of the inner cannula during its insertion and removal from the outer cannula, and an interlocking means for holding the inner cannula within the outer cannula. The interlocking means engage with a connecting means that is attached to the outer cannula.

The present invention may also comprise an obturator which helps guide and support the outer cannula during the outer cannula's insertion into the trachea. The obturator comprises a distal end that is inserted into the outer cannula, a proximal end that is outside of the trachea, and a middle region therebetween which has a horizontal section proximally adjacent to a section having a general curvature. The obturator features wing-like structures which contribute to positioning the obturator when inserted in the outer cannula, and an interlocking means which holds the obturator within the outer cannula. The interlocking means of the obturator engage with a connecting means that is attached to the outer cannula.

Moreover, the present invention may comprise an outer cannula and a removable inner cannula that is designed for insertion into the trachea of a patient having an extra-long stoma. This outer cannula comprises a distal end that is inserted into the trachea, a proximal end that is outside of the trachea, and a lumen therebetween which may have a horizontal section adjacent proximally to a section having a general curvature. The horizontal section of the outer cannula in this embodiment is longer as compared to the horizontal section of an outer cannula designed for a normal stoma. The inner cannula comprises a distal end that is inserted into this extra-long outer cannula, a proximal end that is outside of the trachea, and a middle region therebetween which may have a horizontal section adjacent proximally to a section having a general curvature. The horizontal section of the inner cannula of this embodiment is longer as compared to the horizontal section of an inner cannula designed for a normal stoma. This extra-long inner cannula features a gripping means on its proximal end, which enables ease in handling the inner cannula during its insertion and removal from the extra-long outer cannula, and an interlocking means for holding the inner cannula within the extra-long outer cannula. The interlocking means engage with a connecting means that is attached to the outer cannula.

The present invention may further comprise an obturator which helps guide and support the outer cannula during insertion into the trachea of a patient having an extra-long stoma. The obturator comprises a distal end that is inserted into the extra-long outer cannula, a proximal end that is outside of the trachea, and a middle region therebetween which may have a horizontal section adjacent proximally to a section having a general curvature. The horizontal section of the obturator of this embodiment is longer as compared to the horizontal section of the obturator designed for a normal stoma. This extra-long obturator features wing-like structures which contribute to positioning the obturator when it is inserted in the extra-long outer cannula, and an interlocking means which holds the extra-long obturator within the extra-long outer cannula. The interlocking means of the extra-long obturator engage with a connecting means that is attached to the extra-long outer cannula.

The present invention may also comprise a tracheostomy tube kit for patients having a normal-sized stoma, or a tracheostomy tube kits for patients having an extra-long stoma. These kits comprise the outer cannula, the inner cannula, and the obturator of the invention as disclosed above.

The present invention may comprise a method of inserting a tracheostomy device into the trachea of a patient having a stoma, comprising inserting the obturator of the present invention into the outer cannula of the present invention, inserting the outer cannula/obturator assembly through the stoma and into the airway, removing the obturator, and then inserting the inner cannula of the present invention into the outer cannula.

The present invention may further comprise a method of inserting a tracheostomy tube into trachea of a patient having an extra-long stoma, comprising inserting the extra-long obturator of the present invention into the extra-long outer cannula of the present invention, inserting the extra-long outer cannula/extra-long obturator assembly through the extra-long stoma and into the airway, removing the extra-long obturator, and then inserting the extra-long inner cannula of the present invention into the extra-long outer cannula.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 shows an outer cannula according to one embodiment of the invention.

FIG. 2A shows an outer cannula with a connecting means according to one embodiment of the invention.

FIG. 2B shows a magnified view of a connecting means according to one embodiment of the invention.

FIG. 3A shows an inner cannula according to one embodiment of the invention.

FIG. 3B shows an cross-sectional view of an inner cannula according to one embodiment of the invention.

FIG. 4 shows an inner cannula according to an alternative embodiment of the invention, wherein the inner cannula features a segmented design.

FIG. 5 shows an inner cannula inserted into an outer cannula according to one embodiment of the invention.

FIG. 6 shows the proximal end of an inner cannula according to one embodiment of the invention.

FIG. 7A shows an obturator according to one embodiment of the invention.

FIG. 7B shows a frontal view of an obturator according to one embodiment of the invention.

FIG. 8 shows an obturator according to an alternative embodiment of the invention, wherein the obturator features a segmented design.

FIG. 9 shows an obturator inserted into an outer cannula according to one embodiment of the invention.

FIG. 10 shows the proximal end of an obturator according to one embodiment of the invention.

FIG. 11 shows an outer cannula designed for an extra-long stoma according to one embodiment of the invention.

FIG. 12 shows an inner cannula designed for an extra-long stoma according to one embodiment of the invention.

FIG. 13 shows an obturator designed for an extra-long stoma according to one embodiment of the invention.

### DETAILED DESCRIPTION

In the following description, like reference characters designate like or corresponding parts throughout the figures. Additionally, in the following description, it is understood that such terms as “front,” “back,” “left,” “right,” “transverse,” “longitudinal,” “upward,” “downward,” “superior” and “inferior” and the like are words of convenience and are not to be construed as limiting terms.

The present invention is directed to a dual-cannula tracheostomy device for insertion into the trachea of a patient having a stoma. The device is comprised of an outer cannula and a removable inner cannula, wherein the inner cannula features a gripping means to provide easier handling of the inner cannula during its insertion and removal from the outer cannula, and an interlocking means for stabilizing the inner cannula within the outer cannula.

The present invention is also directed to a tracheostomy device that additionally comprises an obturator. The obturator features wing-like structures to position the obturator when it is inserted into the outer cannula, and an interlocking means which holds the obturator within the outer cannula.

Moreover, the present invention is directed to a tracheostomy device for insertion into the trachea of a patient having an extra-long stoma. The device is comprised of an outer cannula and a removable inner cannula which are both extended in length. The extra-long inner cannula features a gripping means to provide easier handling of the extra-long inner cannula during its insertion and removal, and an interlocking means for holding the extra-long inner cannula within the extra-long outer cannula.

The present invention is directed to a tracheostomy tube device that further comprises an obturator for use with a patient having an extra-long stoma. The obturator is extended in length and features wing-like structures, which position the extra-long obturator in the extra-long outer cannula, and an interlocking means, which holds the extra-long obturator within the extra-long outer cannula.

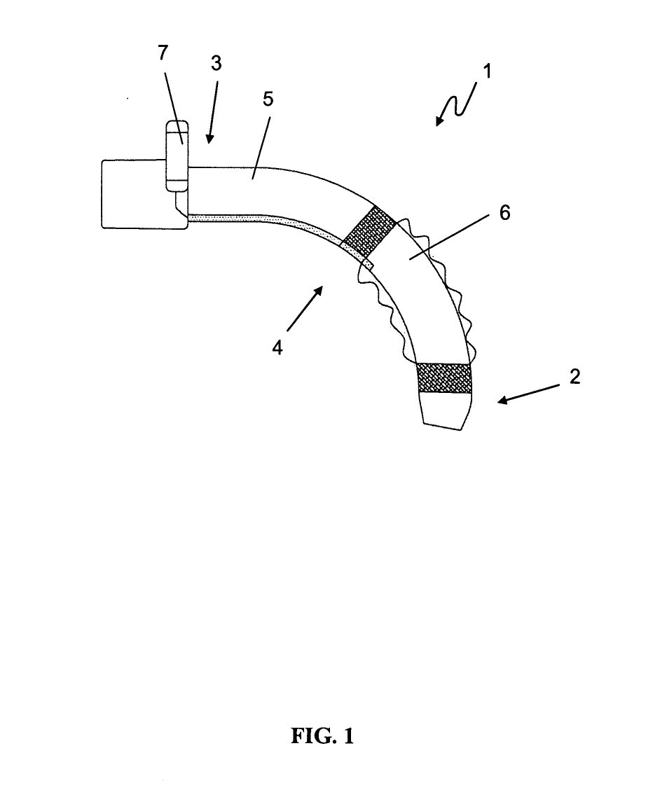

FIG. 1 illustrates a preferred embodiment of an outer cannula 1. The outer cannula comprises a distal end 2, a proximal end 3, and a lumen 4 therebetween. The lumen 4 may comprise a horizontal section 5 and a section having a general curvature 6 designed to fit through the stoma and into the trachea of the patient. The dimensions of the outer cannula 1 are well known in the art that is established for tracheostomy devices (for example, see Hess D R, Respiratory Care 50: 497-510, 2005). The outer cannula can be made of metal or plastic. Exemplified metal materials include silver, stainless steel, and the like, while plastic materials include silicone and, preferably, polyvinyl chloride and the like. Also, the outer cannula is preferably cuffed, as is known in the art. Furthermore, the outer cannula can be adapted to connect to ventilation systems and the like, as is known in the art.

In a preferred embodiment, the outer cannula 1 also comprises a flange 7 on the proximal end, which provides a means to stabilize the outer cannula outside of the patient's body. A connecting means can be attached to the flange to eliminate separation from the ventilation circuit and prevent air leakage. The attachment of the connecting means to the flange may be via methods known in the art, e.g., snap-fit, adhesive, etc.

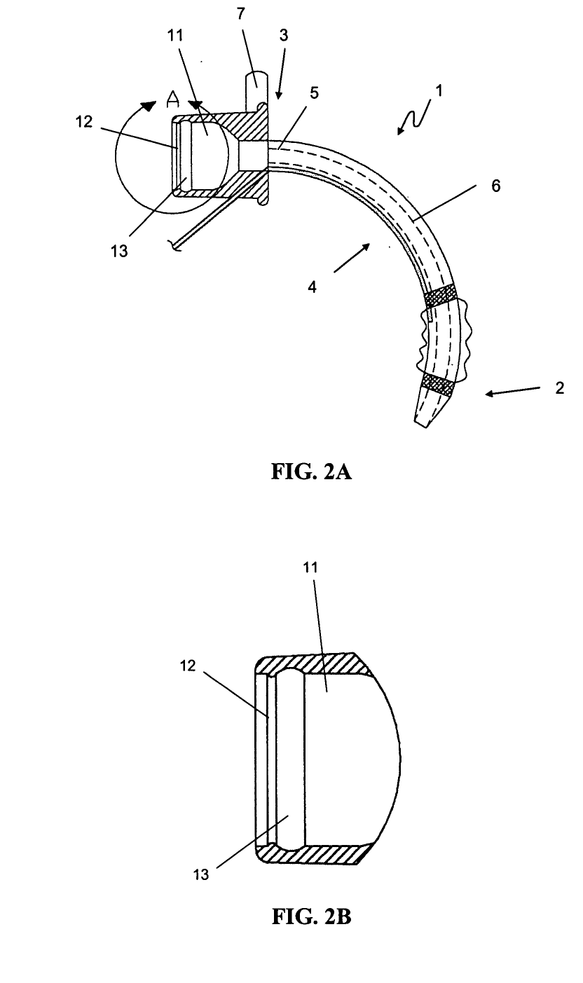

The connecting means is illustrated in FIGS. 2A-2B, and is in the general shape of a cylinder having a lumen with an inner and outer radius located on the proximal end of the outer cannula. In FIGS. 2A-2B, the connecting means 11 comprises ridges 12 and undercuts 13 which can lock in components, e.g., an inner cannula, an obturator, which is inserted into the outer cannula. Notably, undercut refers to a cross-sectional band on the connecting means wherein the internal diameter of the connecting means is slightly greater. The ridge refers to a thin cross-sectional band on the connecting means wherein the internal diameter is slightly less. Preferably, there is a ridge 12 on the proximal end of the connector, and an undercut 13 that is adjacent distally to the ridge 12. In an alternative embodiment, the connecting means may feature a textured surface or protrusions on the inner radial surface.

The connecting means can be comprised of a plastic such as silicone, polyester, polycarbonate, polysulfone, polypropylene, and the like.

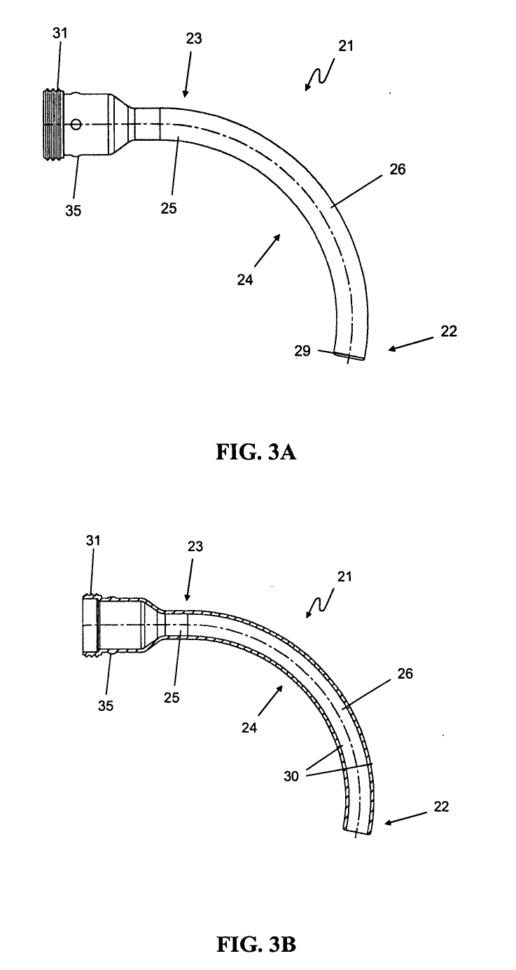

FIG. 3A illustrates a preferred embodiment of the inner cannula 21 which can be inserted into the outer cannula. The inner cannula 21 comprises a tube with a distal end 22, a proximal end 23, and a middle region 24 therebetween. From the proximal end 23 towards the distal end 22, the middle region 24 may contain a horizontal section 25 which is adjacent proximally to a section having a general curvature 26. The inner cannula may further comprise a sealing ring 29 on the distal end 22, which prevents mucus from migrating into the area between the outside of the inner cannula 21 and the inside of the outer cannula 1. The inner cannula can be made of a softer non-toxic plastic, rubber or silicone material.

FIG. 3B shows a cross-sectional view of a preferred embodiment of the inner cannula 21. Notably, the walls 30 of the inner cannula 21 are relatively uniform in width.

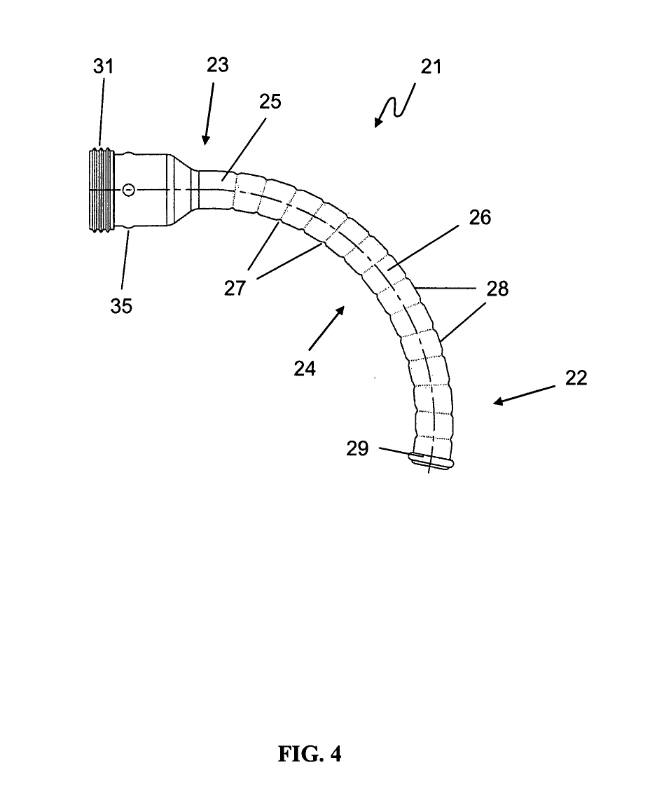

FIG. 4 illustrates an alternative embodiment, wherein the section having a general curvature 26 may have a segmented design featuring thinner wall cross-sections 27, and areas of flat edges 28 located therebetween the thinner wall cross-sections 27. The thinner wall cross-sections 27 will allow the inner cannula 21 to flex during insertion and removal from the outer cannula. The flat edges 28 provide stability during insertion and when the inner cannula 21 is in a fixed position.

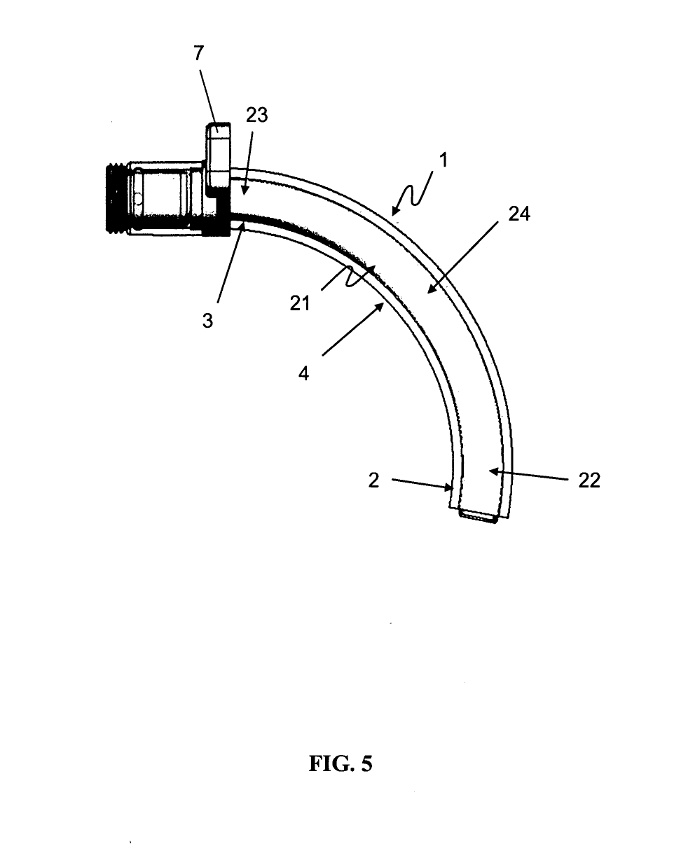

FIG. 5 illustrates the inner cannula 21 inserted into the outer cannula 1. The section having a general curvature 26 of the inner cannula 21 adapts to the shape of the section having a general curvature 6 of the outer cannula when the inner cannula is inserted.

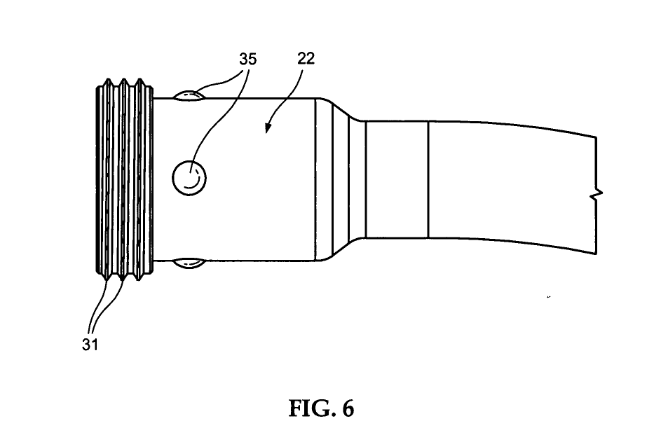

FIG. 6 shows a magnified view of the proximal end 23 of the inner cannula 21, which features a gripping means 31. The gripping means 31 aids in handling the inner cannula during its insertion and removal from the outer cannula 1. The gripping means may comprise different forms, including a textured surface, hooks, and, preferably, ridges. The ridges may be of various shapes, including rounded, squared ridges, trapezoidal, triangular, and the like. Most preferably, the ridges are pyramid-shaped, which is illustrated in the magnified view of the proximal end 23 of the inner cannula 21 shown in FIG. 6. Furthermore, the ridges may be continuous or discontinuous, or distributed uniformly or non-uniformly around the circumference of the inner cannula. Preferably, the ridges are continuous around the circumference of the inner cannula. The number of rows of ridges can vary but there is at least one, and preferably three, row of ridges.

In a preferred embodiment, the inner cannula contains an interlocking means 35 for holding the inner cannula within the outer cannula. The interlocking means 35 engage with the connecting means 11 attached to the flange 7 of the outer cannula I. In other words, when the inner cannula 21 is inserted into the outer cannula 1, the interlocking means 35 on the proximal end 23 of the inner cannula engages with the connecting means 11 on the proximal end of the outer cannula. Preferably, the interlocking means 35 fits securely or locks against the undercut 13 of the connecting means 11. More preferably the interlocking means fits securely within the undercut 13. Even more preferably, the interlocking means 35 extend from the surface of the inner cannula 21, and presses against the inner surface of the undercut 13. Further, the ridge 12 of the connecting means 11 provides an additional means of holding the inner cannula 21 in place when inserted in the outer cannula 1.

The interlocking means 35 may be a textured surface or ridges which can engage in a friction fit with the inner surface of the connecting means. Preferably, the interlocking means are protrusions that extend from the outer surface of the inner cannula 21. The protrusions can be of any shape or form, including cubic protrusions, cylindrical protrusions, pyramidal protrusions, and other prism-shaped protrusions, but most preferably hemispherical protrusions as illustrated in FIG. 6. The number of protrusions on the inner cannula can vary, but is preferably between one and twelve protrusions, more preferably between two and eight protrusions, and most preferably four protrusions. Moreover, the protrusions may be distributed evenly or unevenly around the circumference of the inner cannula, but preferably evenly. There also may be one or more rows of protrusions on the inner cannula, but the number of rows preferably corresponds with the number of undercuts on the connecting means 11. In the preferred embodiment shown in FIG. 6, there is one row of four protrusions distributed evenly around the circumference of the inner cannula, wherein one protrusion is located on each of the superior, inferior, sinister (left), and dexter (right) sides of the inner cannula 21.

The inner cannula 21 and outer cannula 1 may be surface treated internally and externally as described in U.S. patent application Ser. No. 11/440,905, which is herein incorporated by reference. For example, the surface may be treated with a protective polymeric coating using parylene, such as parylene N, parylene C, or parylene D in an applied thickness of 0.00003″ to 0.0001″ and preferably a thickness of 0.00005″.

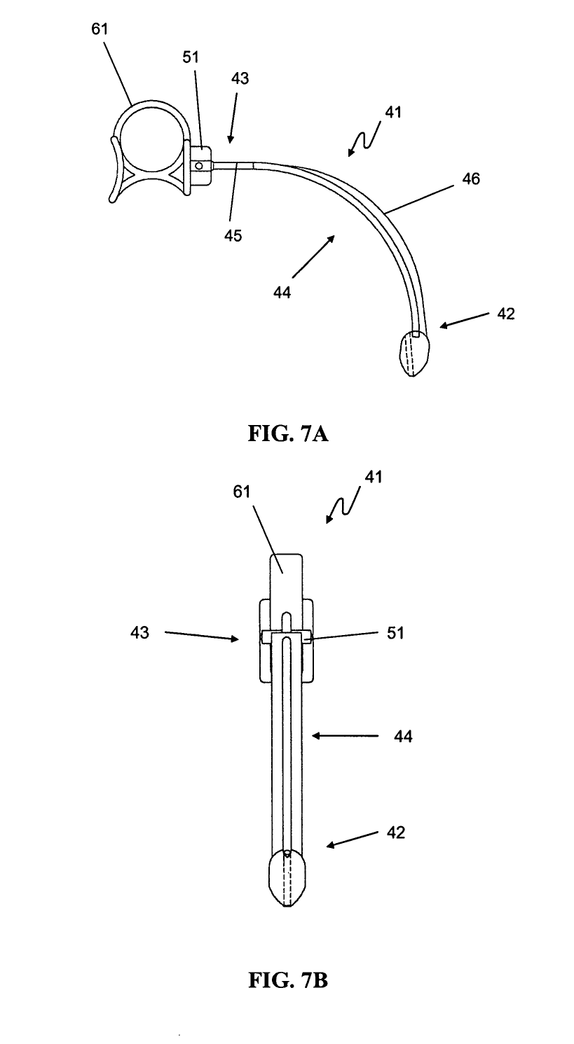

FIGS. 7A and 7B illustrate a preferred embodiment of the obturator 41 which helps guide and support the outer cannula 1 during insertion of the outer cannula into the trachea. The obturator 41 comprises a distal end 42 that is inserted into the outer cannula, a proximal end 43 that is outside of the trachea, and a middle region 44 therebetween. The middle region 44 may comprise a horizontal section 45 which is adjacent proximally to a section having a general curvature 46. The obturator 41 is can be made of a thermoplastic resin such as ABS (acrylonitrile-1,4-butadiene-styrene random or block copolymers), polybutene, polystyrene, and the like, and polyolefins such as polyethylene (high and low density versions), polypropylene, copolymers of ethylene and higher olefins, and the like.

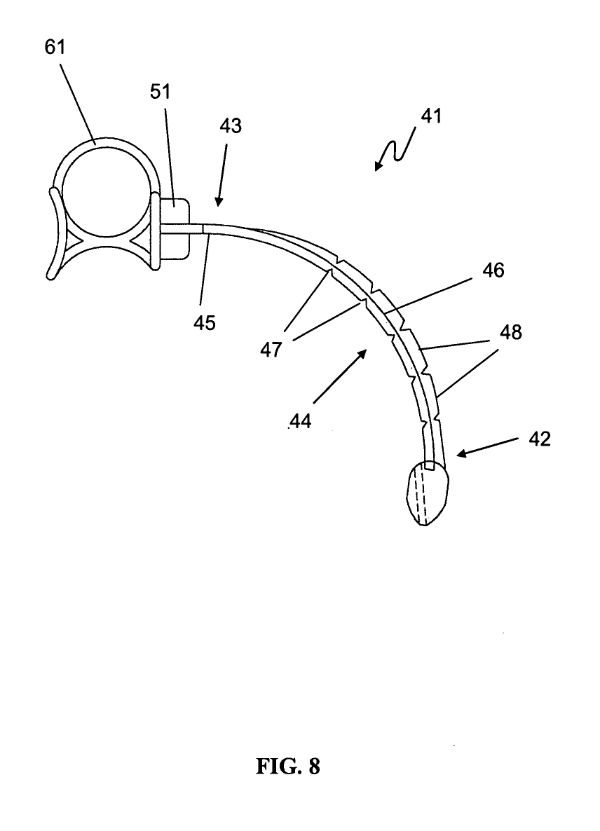

FIG. 8 shows an alterative embodiment featuring a segmented design, wherein the middle region 44, most notably the section having a general curvature 46, is comprised of segments 48 separated by areas of thinner diameter 47. This segmented design provides the obturator with greater flexibility and functionality. The obturator will bend at the areas of thinner diameter 47 to fit the geometry of the section having a general curvature in the lumen of the outer cannula. Preferably, the segments feature ribs on the superior aspect which will prevent the obturator 41 from moving within the outer cannula during insertion of the outer cannula/obturator assembly through the stoma and into the trachea.

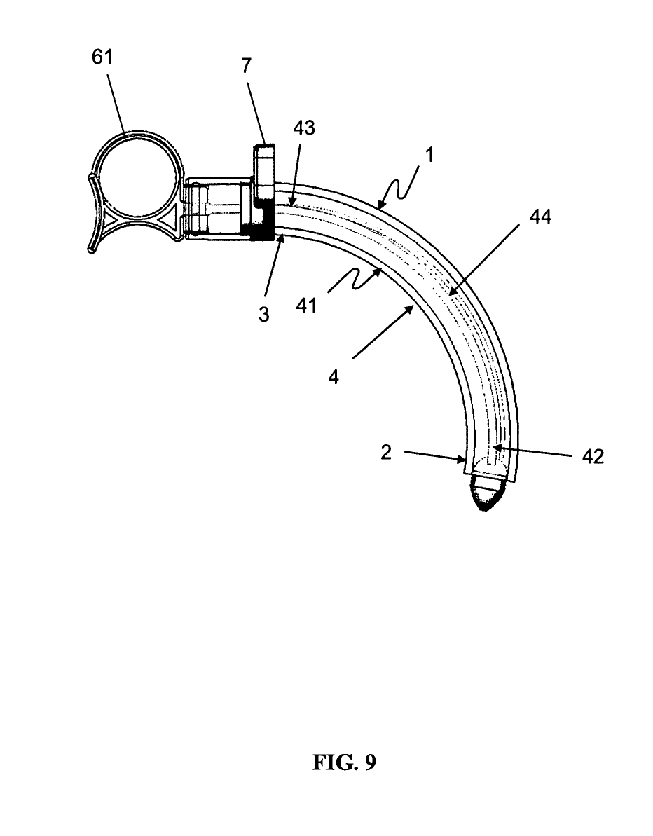

FIG. 9 illustrates the obturator 41 inserted into the outer cannula 1.

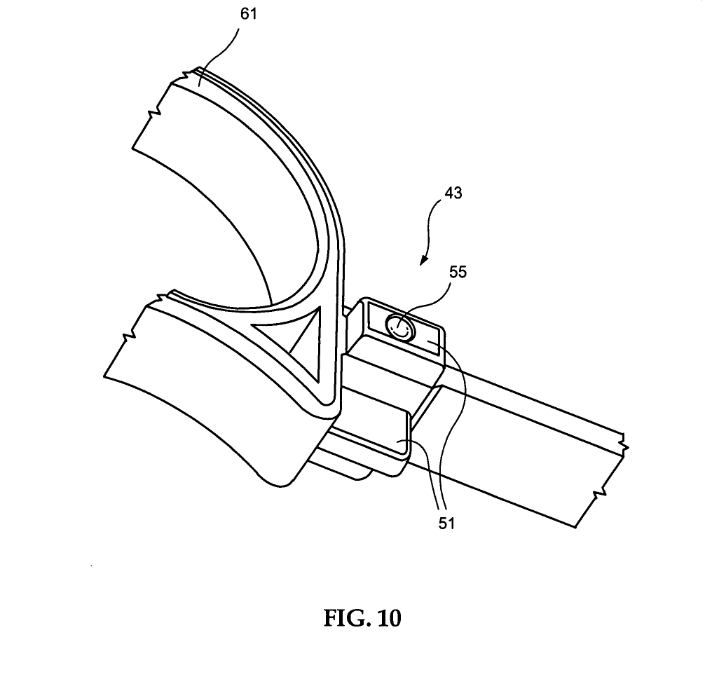

FIG. 10 shows a magnified view of the proximal end 43 of the obturator 41, which features wing-like structures or wings 51. The wings 51 extend outward from the obturator 41 and help position the obturator 41 in the outer cannula 1. More importantly, the wings 51 position the obturator 41 within the connecting means 11 of the outer cannula 1 and hold it steady or centered within the connecting means 11. The wings 51 may be of various shapes, including cubic, spherical, and prism-shapes known in the art, but are preferably rectangular-prism-shaped. Furthermore, the wings 51 may be distributed evenly or unevenly around the edge of the obturator, but are preferably distributed evenly. The number of wings may also vary but are preferably between one and eight, more preferably between two and six, and most preferably four. The most preferred embodiment is shown in FIG. 10, wherein there are four wings 51, and one wing each is on the superior, inferior, sinister (left), and dexter (right) side of the obturator 41.

In a preferred embodiment, the obturator 41 also has an interlocking means 55 which contributes to hold the obturator 41 within the outer cannula 1. The interlocking means of the obturator engage with the connecting means that is attached to the outer cannula. Preferably, the interlocking means 55 fits securely or locks against the undercut 13 of the connecting means 11. More preferably the interlocking means 55 extend radially and engage with the inner surface of the undercut 13 on the connecting means 11. Further, the ridge 12 is an additional means for holding obturator 41 in place.

The interlocking means 55 may be a textured surface or ridges which can engage in a friction fit with the inner surface of the connecting means. Preferably, the interlocking means are protrusions that extend from the outer surface of wings 51 of the obturator 41. The protrusions can be of any shape or form, including cubic protrusions, cylindrical protrusions, pyramidal protrusions, and other prism-shaped protrusions, but most preferably hemispherical protrusions as illustrated in FIG. 10. The number of protrusions on each wing can vary, but is preferably between eight or less protrusions, more preferably four or less protrusions, even more preferably two or less protrusions, and most preferably one protrusion. Moreover, the protrusions may be distributed randomly or in a row or patter on the wing, but is preferably distributed in an ordered pattern, such as in a row, in a triangular shape, in a square, in a circle, etc. In the preferred embodiment illustrated in FIG. 10, there is one protrusion located in the center of the wing 51. The protrusion engages with the inner surface of the undercut when the obturator is inserted in the outer cannula.

The obturator 41 may also comprise a ring finger 61 on its proximal end 42, which helps in handling the obturator during its insertion and removal from the outer cannula. The design of the ring finger is described in U.S. Design Pat. No. 332,140, which is herein incorporated by reference.

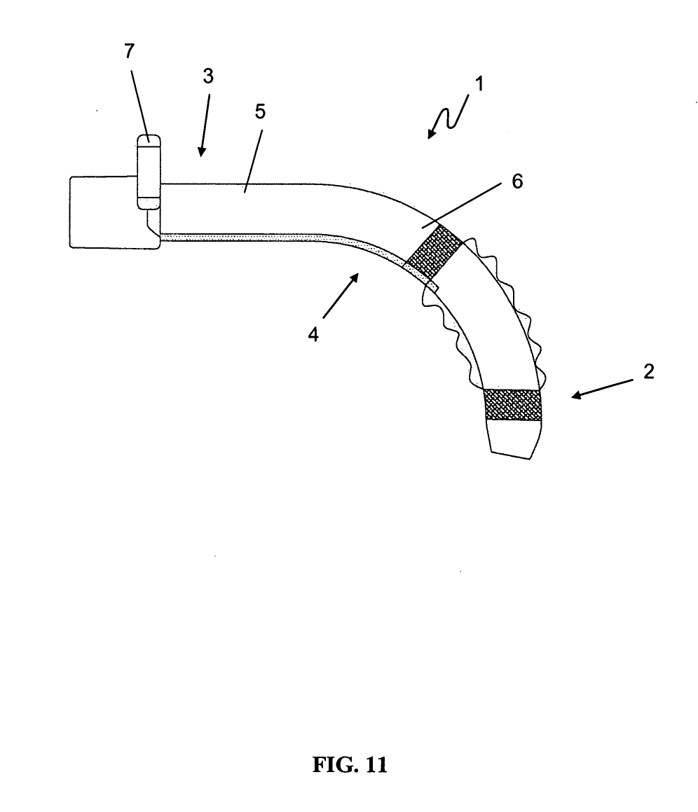

FIG. 11 illustrates a preferred embodiment of an outer cannula 71 designed for insertion into the trachea of a patient having an extra-long stoma. The outer cannula has a distal end 72, a proximal end 73, and lumen 74 therebetween. The lumen 74 may comprise a horizontal section 75 and a section having a general curvature 76 designed to fit through the extra-long stoma and into the trachea of the patient. The dimensions of the outer cannula designed for the extra-long stoma are well known in the art established for tracheostomy devices. However, these dimensions are different than those of outer cannulas designed for regular-length stomas. Most notably, the horizontal region 75 is longer than the corresponding horizontal region 5 that was shown in FIG. 1. Further, the radius of the outer cannula for an extra-long stoma is not constant, and the changes in radius is appropriate for an outer cannula inserted into a patient having an extra-long stoma as known in the art. Also, the outer cannula is preferably cuffed.

The flange, connecting means, and coating described above for use with the outer cannula used in patients having normal-sized stomas are also applicable to outer cannulas used in patients having extra-long stomas.

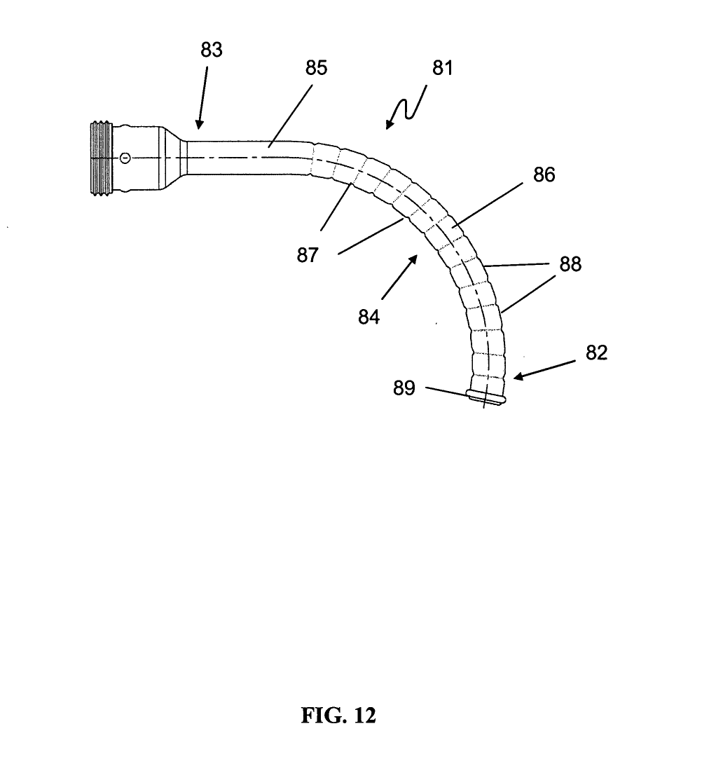

FIG. 12 illustrates a preferred embodiment of the inner cannula 81 which can be inserted into the outer cannula 71 designed for a patient having an extra-long stoma. The inner cannula 81 has a distal end 82, a proximal end 83, and a middle region 84 therebetween. The middle region 84 contains a horizontal section 85 which is adjacent proximally to a section having a general curvature 86. The section having a general curvature 86, may preferably have a segmented design, featuring areas having thinner wall cross-sections 87 and areas of flat edges 88 located therebetween. The thinner wall cross-sections 87 will allow the inner cannula to flex during insertion and removal from the extra-long outer cannula. The flat edges 88 provide stability during insertion of the inner cannula 81 into the extra-long outer cannula 71, and when the inner cannula 81 is in a fixed position within the outer cannula 71. A sealing ring 89 may be located on the distal end 83 of the inner cannula 81.

Compared to the horizontal region 25 of the inner cannula 21 designed for a patient having a normal-sized stoma as shown in FIG. 3, the horizontal region 85 of the inner cannula 81 is designed for a patient having an extra-long stoma and is greater in length in order to appropriately fit inside the extra-long outer cannula 71. The gripping means, interlocking means, and coating described above for use with the inner cannula 21 used in patients having normal-sized stomas are also applicable to inner cannula used in patients having extra-long stomas.

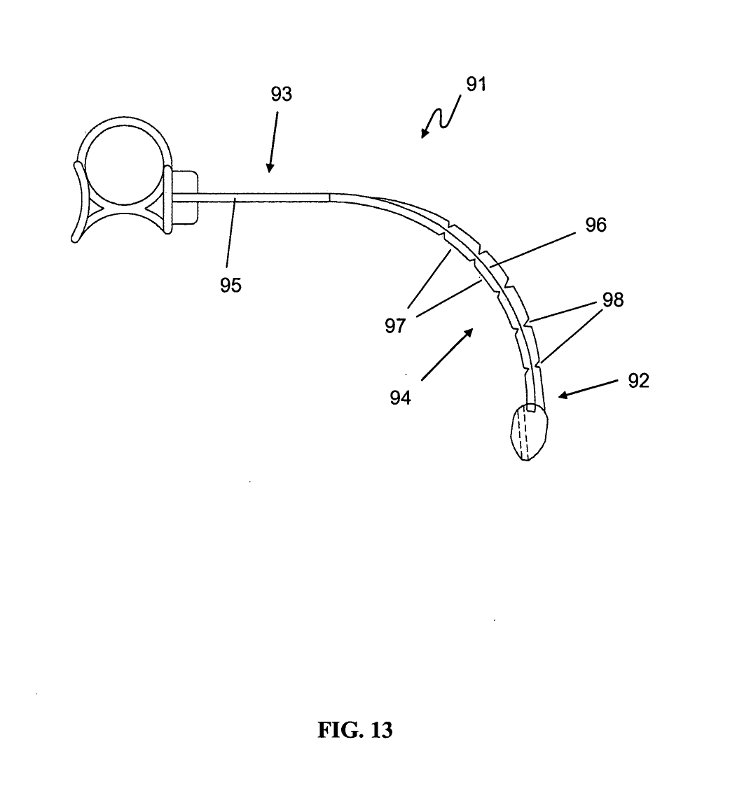

FIG. 13 illustrates a preferred embodiment of the obturator 91 which helps guide and support the outer cannula 71 during insertion into the trachea of a patient having an extra-long stoma. The obturator 91 comprises a distal end 92 that is inserted into the outer cannula, a proximal end 93 that is outside of the trachea, and a middle region 94 therebetween. The middle region 94 may comprise a horizontal section 95 adjacent proximally to a section having a general curvature 96. The middle region 94, most notably the section having a general curvature 96, is preferably comprised of segments 97 separated by areas of thinner diameter 98. This segmented design provides the obturator with greater flexibility and functionality. The obturator will bend at the areas of thinner diameter 98 to fit the geometry of the lumen 74 of the extra-long outer cannula. Preferably, the segments 97 feature ribs on the superior aspect which will prevent the obturator 91 from moving within the extra-long outer cannula 71 during insertion of the extra-long outer cannula/obturator assembly through the extra-long stoma and into the trachea.

Compared to the horizontal region 45 of the obturator 41 designed for a patient having a normal-sized stoma as shown in FIG. 7A, the horizontal region 95 of the obturator 91 is greater in length in order to appropriate fit inside the extra-long outer cannula 71. The wing-like structures, interlocking means, and ring finger component described above for use with the obturator used in patients having normal-sized stomas are also applicable to obturators used in patients having extra-long stomas.

The present invention may also comprise a tracheostomy tube kit for patients having a normal-sized stoma, or a tracheostomy tube kits for patients having an extra-long stoma. These kits comprise the outer cannula, the inner cannula, and the obturator of the invention.

The present invention may comprise a method of inserting a tracheostomy tube into trachea of a patient having a stoma, comprising inserting the obturator of the present invention into the outer cannula of the present invention, inserting the outer cannula/obturator assembly through the stoma and into the airway, removing the obturator, and then inserting the inner cannula of the present invention into the outer cannula.

The present invention may further comprise a method of inserting a tracheostomy tube into trachea of a patient having an extra-long stoma, comprising inserting the extra-long obturator of the present invention into the extra-long outer cannula of the present invention, inserting the extra-long outer cannula /extra-long obturator assembly through the extra-long stoma and into the airway, removing the extra-long obturator, and then inserting the extra-long inner cannula of the present invention into the extra-long outer cannula.

Having thus described in detail preferred embodiments of the present invention, it is to be understood that the invention is not to be limited to particular details set forth in the above description as many apparent variations thereof are possible without departing from the spirit or scope of the present invention.

What is claimed is:

1. A dual-cannula tracheostomy device for insertion into the trachea of a patient having a stoma, comprising:
(i) an outer cannula comprising a distal end for insertion into the trachea, a proximal end for placement outside of the trachea, and a lumen therebetween, wherein the lumen comprises a horizontal section adjacent proximally to a section having a general curvature designed to fit through the stoma and into the trachea of the patient, and wherein a flange is attached to the proximal end of the outer cannula;
(ii) a connecting means attached to the flange of the outer cannula comprising a cylinder with a lumen having at least one undercut; and
(iii) a removable inner cannula comprising a distal end for insertion into the outer cannula, a proximal end that extends to proximal end of the outer cannula, and a middle region therebetween, wherein the middle region comprises a horizontal section that is adjacent proximally to a section having a general curvature designed to fit into the curved region of the outer cannula, and wherein the inner cannula further comprises an interlocking means on the proximal end for locking the inner cannula to the connecting means on the outer cannula, wherein the interlocking means of the inner cannula engages in a friction fit with the undercut of the connecting means.

2. The tracheostomy device according to claim 1, wherein the interlocking means is comprised of at least one protrusion that extends radially from the surface of the inner cannula.

3. The tracheostomy device according to claim 2, wherein the at least one protrusion is hemispherical in shape.

4. The tracheostomy device according to claim 1, wherein the inner cannula further comprises a gripping means proximal to the interlocking means for handling the inner cannula.

5. The tracheostomy device according to claim 4, wherein the gripping means comprises one or more ridges that extend radially from the surface of the inner cannula.

6. The tracheostomy device according to claim 5, wherein the ridges are pyramid-shaped.

7. The tracheostomy device according to claim 1, wherein the connecting means further comprises a ring that provides a friction-fit with the inner cannula.

8. The tracheostomy device according to claim 1, wherein the outer cannula is cuffed.

9. The tracheostomy device according to claim 1, wherein the inner cannula comprises a sealing ring on the distal end of the inner cannula.

10. The tracheostomy device according to claim 1, further comprising an obturator for insertion into the outer cannula.

11. The tracheostomy device according to claim 10, wherein the obturator comprises a distal end for insertion into the outer cannula, a proximal end that extends to the proximal end of the outer cannula, and a middle region therebetween, wherein the middle region comprises a horizontal section that is adjacent proximally to a section having a general curvature designed to fit into the curved region of the outer cannula, and wherein the obturator further comprises an interlocking means on the proximal end for locking the obturator to the connecting means on the outer cannula, wherein the interlocking means of the obturator engages in a friction fit with the undercut of the connecting means.

12. The tracheostomy device according to claim 11, wherein the obturator further comprises wing-like structures, wherein the wing-like structures are prism-shaped and extend radially from the obturator to stabilize the obturator within the connecting means.

13. The tracheostomy device according to claim 12, wherein the wing-like structures are cuboid in shape.

14. The tracheostomy device according to claim 13, wherein the wing-like structures comprises an interlocking means on its outer surface.

15. The tracheostomy device according to claim 14, wherein the wing-like structures comprise an interlocking means on the outer surface facing the connecting means.

16. The tracheostomy device according to claim 15, wherein the interlocking means are at least one protrusion that extends radially from the outer surface facing the connecting means.

17. The tracheostomy device according to claim 16, wherein the one or more protrusions is hemispherical in shape.

18. The tracheostomy device according to claim 10, wherein the obturator comprises a ring finger on the proximal end for handling the obturator.

19. The tracheostomy device according to claim 1, wherein the inner cannula and outer cannula are treated with a protective polymeric coating comprising parylene.

20. A dual-cannula tracheostomy device for insertion into the trachea of a patient having an extra-long stoma, comprising:
(i) an outer cannula comprising a distal end for insertion into the trachea, a proximal end for placement outside of the trachea, and a lumen therebetween, wherein the lumen comprises a horizontal section that is adjacent proximally to a section having a general curvature designed to fit through the stoma and into the trachea of the patient, wherein the horizontal section is elongated for fitting through an extra-long stoma, and wherein a flange is attached to the proximal end of the outer cannula;
(ii) a connecting means attached to the flange of the outer cannula comprising a cylinder with a lumen having at least one undercut; and
(iii) a removable inner cannula comprising a distal end for insertion into the outer cannula, a proximal end that extends to proximal end of the outer cannula, and a middle region therebetween, wherein the middle region comprises a horizontal section that is adjacent proximally to a section having a general curvature designed to fit into the curved region of the outer cannula, and wherein the horizontal section is elongated for fitting through the elongated horizontal section of the outer cannula, and wherein the inner cannula further comprises an interlocking means on the proximal end for locking the inner cannula to the connecting means on the outer cannula, wherein the interlocking means of the inner cannula engages in a friction fit with the undercut of the connecting means.

21. The tracheostomy device according to claim 20, further comprising an obturator for insertion into the outer cannula and said inner cannula and/or said obturator are segmented.

22. The tracheostomy device according to claim 21, wherein the obturator comprises a distal end for insertion into the outer cannula, a proximal end that extends to the proximal end of the outer cannula, and a middle region therebetween, wherein the middle region comprises a horizontal section that is adjacent proximally to a section having a general curvature designed to fit into the curved region of the outer cannula, and wherein the horizontal section is elongated for fitting through the elongated horizontal section of the outer cannula, and wherein the obturator further comprises an interlocking means on the proximal end for locking the obturator to the connecting means on the outer cannula, wherein the interlocking means of the obturator engages in a friction fit with the undercut of the connecting means.

23. A method of inserting the tracheostomy device of claim 10 into the trachea of a patient having a stoma, comprising:
(i) inserting the obturator into the outer cannula of the present invention,
(ii) inserting the outer cannula/obturator assembly through the stoma and into the trachea,
(iii) removing the obturator; and
(iv) inserting the inner cannula into the outer cannula.

24. A method of inserting the tracheostomy device of claim 22 into the trachea of a patient having an extra-long stoma, comprising:
(i) inserting the obturator into the outer cannula of the present invention,
(ii) inserting the outer cannula/obturator assembly through the stoma and into the trachea,
(iii) removing the obturator; and
(iv) inserting the inner cannula into the outer cannula.

25. A kit comprising:
(i) an outer cannula comprising a distal end for insertion into the trachea, a proximal end for placement outside of the trachea, and a lumen therebetween, wherein the lumen comprises a horizontal section adjacent proximally to a section having a general curvature designed to fit through the stoma and into the trachea of the patient, and wherein a flange is attached to the proximal end of the outer cannula;
(ii) a connecting means attached to the flange of the outer cannula comprising a cylinder with a lumen having at least one undercut;
(iii) a removable inner cannula comprising a distal end for insertion into the outer cannula, a proximal end that extends to proximal end of the outer cannula, and a middle region therebetween, wherein the middle region comprises a horizontal section that is adjacent proximally to a section having a general curvature designed to fit into the curved region of the outer cannula, and wherein the inner cannula further comprises an interlocking means on the proximal end for locking the inner cannula to the connecting means on the outer cannula, wherein the interlocking means of the inner cannula engages in a friction fit with the undercut of the connecting means; and
(iv) an obturator comprising a distal end for insertion into the outer cannula, a proximal end that extends to the proximal end of the outer cannula, and a middle region therebetween, wherein the middle region comprises a horizontal section that is adjacent proximally to a section having a general curvature designed to fit into the curved region of the outer cannula, and wherein the obturator further comprises an interlocking means on the proximal end for locking the obturator to the connecting means on the outer cannula, wherein the interlocking means of the obturator engages in a friction fit with the undercut of the connecting means.
